(19) United States
(12) Patent Application Publication
(10) Pub. No.: US 2005/0234015 A1
(43) Pub. Date: Oct. 20, 2005

(54) Low molecular weight heparin salt with triethanolamine useful as therapeutic-antitrombotic agent of local delivery, procedures for preparing them, process for elimination of hygroscopicity of heparin salt, pharmaceutical compositions for local use in antithrombotic therapy and uses therein

(76) Inventors: Victor Bautista Diaz, Luis Guillon (AR); Oscar Eduardo Martinez, Luis Guillon (AR)

Correspondence Address: ZARLEY LAW FIRM P.L.C., CAPITAL SQUARE, 400 LOCUST, SUITE 200, DES MOINES, IA, 50309-2350, US

(21) Appl. No.: 11/024,082

(22) Filed: Dec. 28, 2004

(30) Foreign Application Priority Data
Feb. 4, 2004 (AR) P 04 01 00347

(51) Int. Cl. C08B 37/08; A61K 31/727

(52) U.S. Cl. 514/56; 536/20

(57) ABSTRACT

Low molecular weight heparin salt with triethanolamine useful as a therapeutic-antithrombotic agent for local delivery, having at least 60% of its total mass a molecular weight of less than 8000 Da and an average molecular weight from 4000 to 6000 Da, said salt also having a content of organic sulfur from 6.1 to 7.5 weight % (theoretical 6.8%) and a triethanolamine content from 42.6 to 52.1 weight % (theoretical 47.4 weight %). The processes for preparing the heparin salt, eliminating hygroscopicity of said salt, and pharmaceutical compositions for use in local antithrombotic therapy and its uses are also disclosed.

The present invention refers to a new chemical substance obtained by salification of depolymerized acid polysaccharides, particularly, low molecular weight heparin with the organic base TRIETHANOLAMINE. Moreover, pharmaceutical formulations containing said low molecular weight heparin salt (LMWH) with triethanolamine (TEA) are described for its therapeutic delivery by local route. Moreover, the present invention refers to new process for preparing said low molecular weight heparin salt with triethanolamine. A process for eliminating hygroscopicity of heparin salt, pharmaceutical compositions for local use in antithrombotic therapy and uses therein are also described.

The acid polysaccharides, in general, and particularly heparin, may be depolymerized by well reported processes in U.S. Pat. No. 4,977,250/EUROP. PATENT 268885.

Said documents describe a process for the chemical depolymerization of polysaccharides and, particularly, glycosaminoglycan sulfates, comprising heparin, heparan sulfate and dermatan sulfate, as well as the use of the products which are obtained as antithrombotic agents.

Although the depolymerization reaction must be carried out under highly specific conditions, the process is reproducible in practice at technical scale, allowing the obtainment of depolymerized compounds of doubtless value as antithrombotic agents and well recognized by medical practice (particularly, low molecular weight heparin).

The documents mentioned allow the obtainment, starting from injectable heparin USP grade, of low molecular weight heparin in accordance with the European Pharmacopoeia specifications for its use as injectable antithrombotic agent.

Other processes for the chemical depolymerization of acid polysaccharides, particularly glycosaminoglycan sulfates, are detailed in Argentine Patent 243540, Spanish Patent 2007373, Italian Patent 1224260, Pat. BE8700861 and permit, likewise, to obtain low molecular weight heparin starting from injectable heparin USP/EP grade as raw material.

In any case, the depolymerized products obtained by the above methods are sodium salts of low molecular weight heparin, which is not capable of being dermically delivered as it is a substance of strong ionic nature, of significant molecular weight (in average 4000-6000 Da), low affinity to fats and lipids in general and with a no partitioning into water-octanol.

These strongly ionic characteristics of low molecular heparin (and even more, of the starting heparin, due to its higher molecular weight) have severely limited the use of this substance as local delivery antithrombotic agent and, in fact, to date there are no pharmaceutical preparations which are known to contain low molecular weight heparin for local use, for the alleviation o prevention of varicose veins, hemorrhoids, traumatic or post-surgical hematomas (plastic surgery or facial reconstruction surgery).

The present invention describes in detail a new compound, low molecular weight heparin salt with triethanolamine, as well as alternative processes for its preparation, wherein triethanolamine is used in order to increase the lipophilicity of the subject acid polysaccharide, allowing its passage through the skin, by the formation of a stable salt with the polysaccharide, replacing an inorganic counterion which always salifies the heparin and which is preferably selected from sodium, potassium, calcium, magnesium and ammonium, thereby considerably reducing its hydrophilicty and increasing its lipophilicity. The inorganic counterion most preferably used is sodium.

It is inferred therefrom that the low molecular weight heparin structure itself, is not altered by the salification process with triethanolamine (TEA) and, therefore, it does not suffer any variation on its measurable biological properties (anti Xa, anti IIa), as not being the corresponding stoichiometric variation.

For research purposes, the low molecular weight heparin salts were prepared and tested, particularly the diethylamine, triethylamine, tributhylamine, monoethanolamine, diethanolamine and choline salts.

In all cases, triethanolamine (TEA) was the chosen organic base for the carrying out of the invention, due to its recognized use in pharmaceutical and cosmetic dermal preparations (as well as in hair care products) for producing no dermal irritation and for greatly allowing skin penetration of the acid polysaccharide salified with said triethanolamine.

The salt obtained from the reaction of the triethanolamine (TEA) with the low molecular weight heparin (LMWH) is an amber colored substance, with a waxy appearance, extremely difficult to manipulate due to its high hygroscopicity. These characteristics of the LMWH-TEA salt make extremely difficult its precise dosage in the preparation of the pharmaceutical end products (creams, gels, ointments).

Therefore, another characteristic of the present invention is the lyophilization of said LMWH-TEA salt (as it is obtained from its obtainment processes, in aqueous solution) in the presence of Mannitol or Sorbitol. Both monosaccharide polyalcohols are chemically indifferent to the said LMWH-TEA salt as they do not contain aldehyde groups (potential or not) which may react with the amine TEA providing substances like SCHIFF bases, or other type of parasitic reaction, they do not present dermal irritation, they are biologically compatible and chemically stable, as well as commercially available.

Particularly, it was experimentally found that in the end solid lyophilizate, a very adequate ratio of LMWH-TEA/Mannitol is 60:40 weight by weight, which offers a non-hygroscopic lyophilized solid, well manipulated, stable and susceptible of being well dosed by weighing, in the preparation of pharmaceutical products.

Therefore, the detailed characteristic of the present invention includes concepts and methods for reducing the hygroscopicity of the LMWH-TEA salt by lyophilization on a inert carrier (preferably Mannitol or Sorbitol) chemically indifferent and biologically compatible.

The drawings which illustrate the present invention represent:

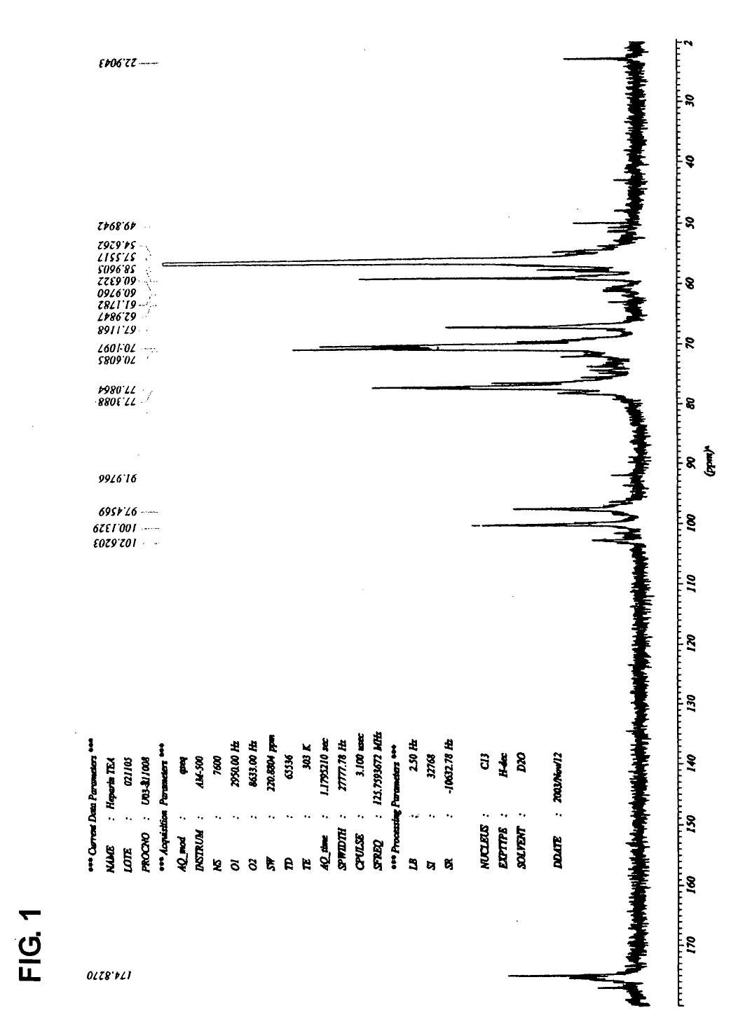

FIG. 1 shows a NMR spectrum (13C) of the LMWH-TEA.

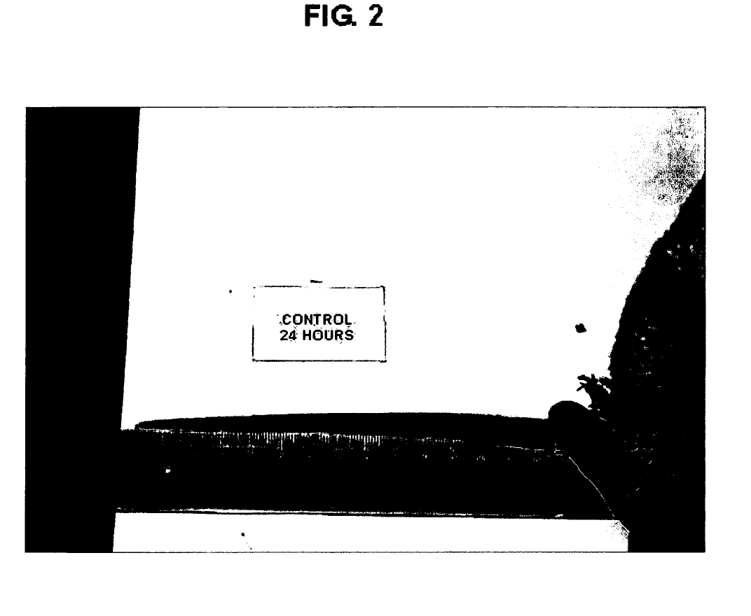

FIG. 2 shows the tail of a sample rat following 24 hours of the application of an injection of k carrageenan.

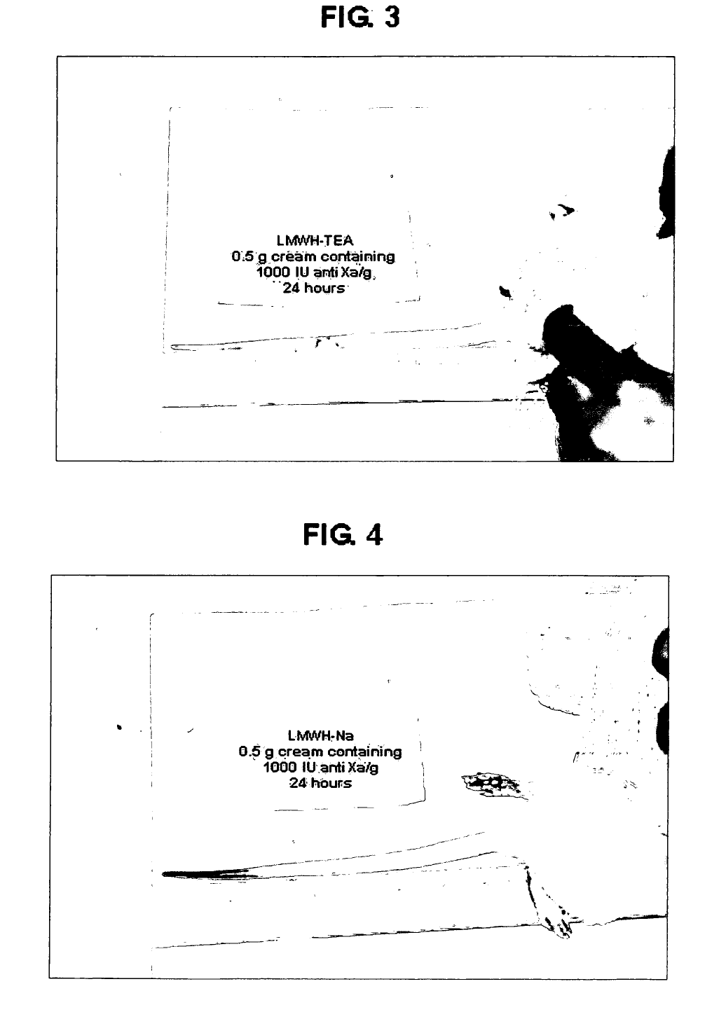

FIG. 3 represents the tail of a rat following 24 hours of the application of an injection of k carrageenan treated with 0.5 g of the cream I which is defined below.

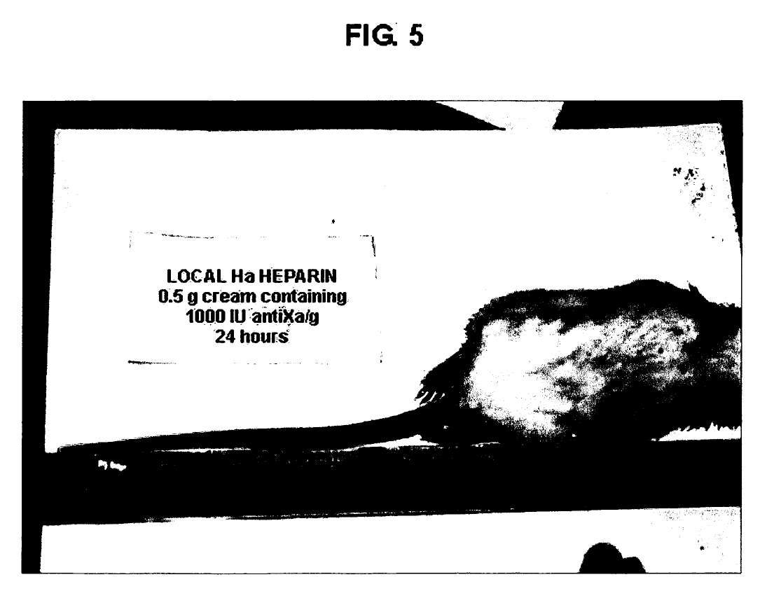

FIG. 4 represents the tail of a rat following 24 hours of the application of an injection of k carrageenan treated with 0.5 g of the cream 11 which is defined below.

FIG. 5 represents the tail of a rat following 24 hours of the application of an injection of k carrageenan treated with 0.5 g of the cream III which is defined below.

The following examples, which are not limited by the generality of the application, describe processes and products obtained.

### EXAMPLE 1

Depolymerization of Heparin

100 g of injectable heparin USP/EP grade are dissolved in distilled water, in a sufficient amount to obtain 500 ml of 20% solution.

The solution is placed in a four entrance balloon provided with stirring, pH-meter electrode, heating and reflux refrigerant. The aqueous solution was warmed at 94-96° C. and then a 1 M hypochlorous acid solution is added at a temperature of 0/5° C. (prepared by fitting to pH=6 with HCl of 80 g/l sodium hypochlorite solution previously refrigerated). 290 ml of 1M solution of HClO is added in a 60 minute reaction, while keeping the pH constant from 5.0 to 6.0 adding HCl or NaOH in aqueous solution, as appropriate.

After the 60 minute reaction has been completed, 3% sodium chloride is added and the reaction product precipitates quickly adding 2 volumes of ethanol under vigorous stirring, thereby producing the immediate blockade of the depolymerization reaction.

Salification with TEA

Once the hydroalcoholic supernatant is decanted, the precipitated polysaccharide (LMWH sodium salt) is dissolved in distilled water in a sufficient amount to provide a 5% w/v solution. The pH is regulated to 5.0-5.5 with acetic acid solution and the solution is subjected to diafiltration (cut off 5000 Da) versus 5% TEA solution in distilled water (pH adjusted to 5.0-5.5 with acetic acid). 20 vol of triethanol ammonium acetate are thereby used.

Subsequently, the diafiltered solution is subjected to diafiltration with 10 volumes of distilled water to remove the free TEA residues from the solution.

The solution is discharged from the ultrafilter and lyophilized, providing 140 g waxy, light amber, highly hygroscopic product, that is the heparin salt of low molecular weight with triethanolamine, which presents the following comparative analysis:

| | Injectable Heparin | LMWH-TEA |
|---|---|---|
| EP Strength | 182 iu/mg | — |
| anti Xa Strength | — | 51 iu/mg |
| Anti IIa strength | — | 24 iu/mg |
| Sulfur (S) | 11.7% | 6.8% |
| S/COO⁻ Ration | 2.1 | 2.1 |
| Triethanolamine | — | 49% |
| Sodium (Na) | 11.0% | 19 ppm |
| pM (HPSEC) | 13500 Da | 5400 Da |
| <8000 Da | — | 82% |
| 3000-8000 Da | — | 53% |
| <3000 Da | — | 25% |

Note:

The anti Xa and anti IIa biological strengths were measured according to the European Pharmacopoeia EP2002; “Low Molecular Mass Heparin”, as well as the mean molecular weights (weighed average Mw) and the molecular weight distribution. Moreover, the standards and gages are those strictly cited in the mentioned literature. It is noted that in the run buffer for the chromatographic test by HPLC (Na₂SO₄, 0.2M pH 5.0) the TEA is replaced by N^{a+} whereby the declared value is that of the sodium polysaccharide fraction and not the one of the LMWH-TEA salt.

### EXAMPLE 2

Depolymerization of Heparin

100 g of injectable heparin USP/EP grade are dissolved in distilled water, in a sufficient amount to provide 500 ml of 20% w/v solution and it is transferred to a balloon provided with heating, stirring and refrigerant at reflux.

A reagent comprised of 20 ml of 30% H₂O₂ and 1 ml of aqueous solution of 1.5% Fe SO₄.7H₂O is warmed at 80° C. and is added under stirring.

Heating and stirring are continued for a 40 minute period, 2% NaCl is added and the reaction is immediately stopped precipitating the depolymerized polysaccharide by addition of 2 vol. of ethanol.

The obtained precipitate is dissolved in distilled water in a sufficient amount to provide a 10% solution and this solution is introduced in a chromatografic column of 1L capacity (Ø 5.2 cm×50 cm height) containing ionic exchange resin Amberlite® IR-120 (Rohm Haas) (phase H⁺) , at a flux of 0.42 cm³/cm² X minute. The acid eluant is collected onto an ice bath vessel with pH adjustment to 5.0-5.5 value adding dropwise triethanolamine NF grade. Once the passage is completed, the column is washed with distilled water until neutral eluant is obtained. Washings and eluants were collected and pH was adjusted to pH 5.0-5.5 by the addition of triethanolamine.

It is lyophilized, obtaining 156 g of the heparin salt of low molecular weight with triethanolamine, LMWH -TEA salt, highly hygroscopic, waxy and amber.

The following is a comparative analysis:

| | Injectable Heparin | LMWH-TEA |
|---|---|---|
| EP Strength | 180 iu/mg | — |
| anti Xa Strength | — | 50 iu/mg |
| anti IIa Strength | — | 24 iu/mg |
| Sulfur | 11.9% | 6.8% |
| S/COO⁻ Ratio | 2.1 | 2.1 |
| Triethanolamine | — | 50% |
| Sodium | 12.0% | 280 ppm |
| Mw (HPSEC) | 12000 Da | 4800 Da |
| <8000 Da | — | 81% |
| 3000-8000 Da | — | 46% |
| <3000 Da | — | 35% |

The same explanations of Example N^{o} 1 apply to this example.

### EXAMPLE 3

Lyophilization in the Presence of Mannitol: Solving the Problem of Hygroscopicity Inherent to LMWH-TEA.

18.6 g of LMWH-TEA obtained in Example N^{o} 2 are dissolved in distilled water in a sufficient amount to provide 125 ml of 15% w/v solution. 12.40 g of mannitol USP are added under stirring, which is very well dissolved at room temperature, if the initial solution does not have a concentration higher than 15%, as in this case.

The solution is lyophilized to provide a crystalline dried powder, that can be subjected to grinding, of highly reduced hygroscopicity. The milled powder is flowable and does not become clotted.

The following Table of analysis is given for comparative purposes:

| | | LMWH-TEA/ |
|---|---|---|
| | LMWH-TEA | MANNITOL |
| anti Xa Strength | 50 iu/mg | 30 iu/mg |
| anti IIa Strength | 24 iu/mg | 14 iu/mg |
| Sulfur | 6.8% | 4.2% |
| S/COO⁻ Ratio | 2.1 | 2.1 |
| Triethanolamine | 50.0% | 30.0% |
| Sodium | 280 ppm | 170 ppm |
| Mw (HPSEC) | 4800 Da | 4800 Da |
| <8000 Da | 81% | 81% |
| 3000-8000 Da | 46% | 46% |
| <3000 Da | 35% | 35% |
| Mannitol | — | 40% |

### EXAMPLE 4

Depolymerization of Fractions of Low Strength Heparin

In preparing injectable heparin, byproducts are obtained containing heparins of low anticoagulant activity together with dermatan sulfate and chondroitin sulfate at varying concentrations. The depolymerized products of these byproducts may be salified with triethanolamine and used as antithrombotics locally.

This example, is not a limitation of the generality of the cases, starts from a fraction of low strength heparin, characterized by the following analysis (0213409IIA)

| | |
|---|---|
| USP Strength: | 45 iu/mg |
| Specific Optic Rotation: | +41° |
| Composition (Electrophoresis): | 12% “Slow moving” (SM) Fraction |
| | 77% “Fast moving” (FM) Fraction |
| | 11% Dermatan Sulfate |

The electrophoretic run was carried out according to Cappelletti, R et al, Anal. Biochem. 99 311-315 (1979).

50 g of the above fraction are dissolved in distilled water in a sufficient amount to provide 250 ml of 20% solution. The solution is placed into a 500 ml capacity balloon equipped with thermometer, heating, stirring and refrigerant at reflux. It is warmed to 95-98° C. and then 580 ml of hypochlorous acid solution 1M are added (obtained by pH adjustment to pH 6 of 80 g/l NaClO solution with hydrochloric acid at a temperature from 0-5° C.) for 60 minutes, while keeping pH constant at all times from 5.0-6.0 values adding a hydrochloric acid or sodium hydroxide solution, as appropriate. Once the addition of the solution is completed, it is precipitated adding two volumes of ethanol. The depolymerized polysaccharide which constitutes the precipitate is dissolved in distilled water in a sufficient amount to provide a 5% solution and pH is adjusted to 5.0-5.5 value with acetic acid solution. The clear solution thus obtained is subjected to a diafiltration process in a appropriate ultrafilter, equipped with a cassette of UF cut off 1000 Da, versus 5% solution of triethanolamine in water, of pH adjusted to 5.0-5.5 with acetic acid.

20 volumes of dialyzer solution are thereby passed and once this step is completed, the diafiltration is continued with the passage of 10 vols. of distilled water to remove free TEA from the concentrate retained by the UF membrane. The solution is discharged from the UF and lyophilized providing 42 g of a product of waxy consistency and amber colored, highly hygroscopic, which is the triethanolamine salt of the initial depolymerized heparinic fraction.

The product presents the following comparative analysis.

| Anti Xa strength | 73 iu/mg (EP Method) |
|---|---|
| Anti IIa strength | 13 iu/mg (EP Method) |
| Sulfur | 4.8% |
| S/COO⁻ Ratio | 1.7 |
| Electrophoresis | 90% FM Fraction |
| 10% Dermatan sulfate | |
| Triethanolamine | 49.0% |
| MW (HPSEC) | 5060 Da |
| <8000 Da | 82.0% |
| <3000 Da | 30.0% |
| <2000 Da | 14.0% |
| Specific Optic Rotation | +20° |

Characterization of the LMWH-TEA Substance

1) Theoretic Content of TEA in the LMWH-TEA Salt

Heparin is defined as a family of polysaccharides, which chains are formed alternating residues of uronic acid and D-glucosamine bonded one to the other by 1-4 bonds and with several grades of sulfating. The uronic acid residue is L-iduronic or D-glucuronic and the glucosaminic residue may be N-sulfated or N-acetilated.

(Casu, B; “Methods of Structural Analysis” in “Heparin, Chemical and Biological Properties, Clinical Applications”, Lane, D; Lindhal, U., Eds; E. Arnold (1989) 25-49)

Therefore, only one approximate formula can be obtained for the disaccharide unit (which does not really exist, due to the molecular microheterogenicity of the heparin). The following formula is in good agreement with the experimental analytical data of an injectable heparin which meets the USP-EP specifications:

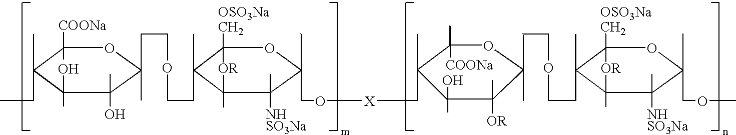

wherein: R=NaSO₃ or H
m=1 and n≧4

m+n=approx. 9
Molecular formula of the unit disaccharide (for R═H)
   C₁₂H₁₅O₁₆NS₂Na₃
Molecular weight of the unit disaccharide (for R═H): 562

The depolymerization reaction, which enables obtaining low molecular weight heparin starting from injectable heparin, does not introduce marked modifications throughout the polysaccharide chain, except for the molecular weight reduction and the properties related thereto (for instance, viscosity).

Therefore, in the present theoretical calculation of the TEA content in LMWH-TEA, we will accept that the unit disaccharide remains intact with the sole change of Na⁺ for the ion Triethanolammonium.

Thus, 100 g of LMWH sodium salt contain, according to the above scheme, 12,1 g Na⁺ (0.527 gram atom of Na⁺) which shall be replaced, in the composition of the triethanolamine salt, by 0.52 mol triethanolamine (under the form of 0.52 mol of the ion triethanolammonium).

The molecular weight of the TRIETHANOLAMMONIUM ion is 150.2 therefore 0.527 mol triethanolammonium=150.2×0.527=79.15 g

In the ionic exchange:

100 g LMWH Na lose 12.1 g Na⁺ and gain 79.15 g of triethanolammonium, producing 167.0 g of LMWH-TEA.

Therefore, the theoretical content of

$$TEA = \frac{79.15 \times 100}{167} = 47.40 \%$$

2) Theoretical Content of S in LMWH-TEA

According to the unit disaccharide accepted as starting point, the % S=11.4%, in good accordance with the analytical data that presents an injectable heparin which meets the USP-EP specifications.

In the depolymerization of the injectable heparin and in the subsequent salification of the depolymerized product, with TEA, S is not lost, so the theoretical data of S in LMWH-TEA should be:

$$\frac{11.4 \times 100}{167} = 6.8 \% \qquad theoretical S .$$

3) Theoretical Calculation of the Biological Strengths in LMWH-TEA

Low molecular weight heparins (sodium salt) obtained by radical fragmentation methods of injectable heparin, such as those cited in European Pat. 268885, U.S. Pat. No. 4,977,250, present an anti Xa strength≅90 iu/mg and an anti IIa strength≅40 iu/mg.

Taking into account the dilution which undergo these values because of the molecular weight increase of the unit disaccharide when eliminating Na⁺ and salifying with triethanolamine, we will have that:
100 g LMWH-Na=167 g LMWH-TEA
therefore,

$$\begin{matrix}theoretical anti Xa strength == \frac{90}{1.67} = 54 iu . / mg \\ theoretical anti IIa strength == \frac{40}{1.67} = 24 iu / mg\end{matrix}$$

4) Comparison of Theoretical Analytical Data and Formulae of the Unit Disaccharides

| | INJECTABLE | | |
|---|---|---|---|
| | HEPARIN | LMWH-Na | LMWH-TEA |
| Sulfur | 11.4% | 11.4% | 6.8% |
| S/COO⁻ | 2.1 | 2.1 | 2.1 |
| Na⁺ | 12.1% | 12.1% | — |
| Triethanolamine | — | — | 47.4% |
| MW (HPSEC) | 12000-13500 | 4000-6000 | 4000-6000 |
| Molecular Formula | C₁₂H₁₅O₁₆NS₂Na₃ | C₁₂H₁₅O₁₆NS₂Na₃ | C₁₂H₁₅O₁₆NS₂[NH(CH₂CH₂OH)₃] |
| of the disaccharide | | | |
| residue | | | |
| Molecular weight of | 563 | 563 | 944 |
| the disaccharide | | | |
| residue | | | |

5) Comparison of Theoretical and Experimental Data in LMWH-TEA

| | Theoretical | Experimental |
|---|---|---|
| Triethanolamine | 47.4% | 48-50% |
| Sulfur | 6.8% | 6.8% |
| S/COO | 2.1 | 2.1 |
| anti Xa | 54 iu/mg | 50-51 iu/mg |
| anti IIa | 24 iu/mg | 24 iu/mg |

6) Spectroscopic Characterization (13C-NMR) of LMWH-TEA

The sample of low molecular weight heparin, triethanolamine salt, was dissolved in H₂O:D₂O:1:1 at conc. of 180 mg/ml. The spectrum of nuclear magnetic resonance (13C) was recorded on a spectrometer (13C) BRUCKER AM-500 (13C-125 MHz), as illustrated in FIG. 1.

Signals of triethanolamine are observed at 56.1 and 56.3 ppm, subsequently determining the relative concentration of iduronic acid 2 sulfate with regard to total uronic acid based upon relative integration of signals of C1 to 100.1 and 102.6 ppm (sulfated and free respectively). In the same manner, total glucosamine N-sulfate/glucosamine (59; 54.6 y 53 ppm) was determined. Data obtained are plotted as follows:

| | Idu 2OS | Glu NS | Glu 6OS | Glu N/Idu H |
|---|---|---|---|---|
| LMWH-TEA | 75% | 85% | 77% | 1:1.67 |

7) Analytical Techniques Used
The anticoagulant strength of injectable heparin was determined according to European Pharmacopoeia 2002 (EP strength) and to USP 26 (USP strength)
Anti Xa and anti ha strengths were determined according to the literature of low molecular weight heparins (European Pharmacopoeia 2002)
Distribution of molecular weights and mean ponderal molecular weight were determined according to the literature of low molecular weight heparins (European Pharmacopoeia 2002), therefore, in the run buffer, TEA is replaced by Na⁺ and the data obtained then pertain to polysaccharide sodium salt and not to TEA salt.
Triethanolamine is dosed by potentiometric titration.

8. -Experimental Pharmacology; Efficiency Test.

Compound LMWH-TEA according to the present invention was subjected to pharmacological tests to verify its efficacy.

With such criteria, Wister rats (female) raised in the laboratory and sanitarily controlled were used as test mammal.

Particularly, the thrombosis model developed consists of generating an experimental thrombosis in the tail of the test rat by intravenous injection (right caudal vein) of a thrombogenic substance such as κ-carrageenan. Without treatment, the tail of the animal totally forms thrombus without remission over time and ends up with necrosis after 3-4 days.

However, local treatment with a cream of local use containing LMWH-TEA at the rate of 1000 u antiXa/gram of cream shows a highly significant restitutive activity much higher than the one demonstrated by local heparin and LMWH sodium salt when administrated under same conditions.

Particularly, this test was carried out according to the general outlines in Beckmeier et al, Agents and Actions, 16, 446 (1985), as follows:

a. Animals: female rats of 200-300 grams weight.

b. Thrombosing Solution: κ carrageenan Type II solution (Sigma C 1263) 1 mg/ml in sterile physiologic solution. 1 ml /kg of animal (right caudal vein of the tail) is injected (0.25 ml).

c. Thrombus induction: a tourniquet is placed near the tail base (15 cm from the end thereof) which was previously shaved. After 1 minute a thrombosing solution is injected and the animal tail is immediately immersed for 30 seconds in ice water, water is removed, it is dried and the tourniquet is kept for an additional 30 seconds. Then, the animal is kept for 2 minutes in a chamber at 10° C.

d. Efficiency test: Then, an experimental cream (0.5 g) containing 1000 units anti Xa/gram of LMWH TEA, sodium LMWH and 1000 IU/gram of local heparin respectively, is administered along the tail that has suffered an infarct to each group of animals (three per cream preparation). Another three animals do not receive treatment and are considered controls.

The animals were controlled 2, 4, 18, 24 and 72 hours post-injection of κ-carrageenan measuring in each case the portion of tail that has suffered an infarct in millimeters (mm).

The results which were obtained were the following:

| | Time | | | | |
|---|---|---|---|---|---|
| | 2 h | 4 h | 18 h | 24 h | 72 h |
| Control | 134 | 134 | 134 | 134 | 133 |
| I | 18 | 13 | (*) | — | — |
| II | 44 | 44 | 43 | 40 | 39 |
| III | 110 | 100 | 92 | 92 | 86 |

Cream I: LMWH-TEA 1000 IU anti Xa/gram
Cream II: LMWH Na 1000 IU anti Xa/gram
Cream III: Local Heparin 1000 IU/gram
(*) very slight stain

For clarity reasons, FIGS. 2 to 5 are attached which are taken 24 h post-injection in each treated group and controls.

In this test, creams for local use were prepared according to the following formula, which is not limiting of the method:

| | Cream I | Cream II | Cream III | |
|---|---|---|---|---|
| Glyceryl | 100 g | 100 g | 100 | g |
| monostearate | | | | |
| Dodecyl oleate | 60 g | 60 g | 60 | g |
| Ketostearyl | 60 g | 60 g | 60 | g |
| alcohol | | | | |
| Ketomacrogol | 30 g | 30 g | 30 | g |
| LMWH-TEA | 10⁶ anti Xa | — | — | |
| LMWH Na | — | 10⁶ anti Xa | — | |
| Local Heparin | — | — | 10⁶ | IU |
| Nipagin | 1.6 g | 1.6 g | 1.6 | g |
| Nipasol | 0.4 g | 0.4 g | 0.4 | g |
| Sterile distilled | 1000 g | 1000 g | 1000 | g |
| water q.s. | | | | |

The test enables the documentation of the marked efficacy of the dermal preparation identified as Cream I (LMWH-TEA), over the other two creams, with the total abatement of the symtoms of thrombosis being observed after 18 hours.

1. Heparin salt of low molecular weight with triethanolamine useful as therapeutic-antithrombotic agent for local administration, characterized in that at least 60% of its total mass has a molecular weight of less than 8000 Da and an average molecular weight from 4000 to 6000 Da, said salt also having a content of organic sulfur from 6.1 to 7.5 weight % (theoretical 6.8 weight %) and a triethanolamine content from 42.6 to 52.1 weight % (theoretical 47.4 weight %).

2. Process for preparing heparin salt of claim 1, characterized in that it comprises the removal by diafiltration of the inorganic counterion of low molecular weight heparin with aqueous solution from 4 to 6% of triethanolamine at pH from 5 to 5.5.

3. Process of claim 2, characterized in that the inorganic counterion is preferably selected from sodium, potassium, calcium, magnesium and ammonium.

4. Process of claim 2, characterized in that the low molecular weight heparin is obtained by thermolytic decomposition of the hypochlorous acid in 1 M aqueous solution at a temperature from 0 to 5° C. on the 15-25% aqueous solution of injectable heparin, at pH from 4 to 7 and a temperature from 70 to 100° C. at reflux.

5. Process of claim 2, characterized in that the low molecular weight heparin is obtained through the radical decomposition of hydrogen peroxide catalized by ferrous ions over the injectable heparin, being the concentration of ferrous ion from 10-100 ppm, the concentration of hydrogen peroxide in a molar ratio H₂O₂/polysaccharide from 6-15, pH from 4 to 7 and a temperature from 60 to 120° C.

6. Process for preparing the heparin salt of claim 1, characterized in that it comprises removing the inorganic counterion of the low molecular weight heparin treating with ion exchange resin in acid phase and subsequent neutralization of the acid polysaccharide eluted with base triethanolamine (99%) adjusting pH of the eluate from 5.0 to 5.5 at a temperature from 15 to 25° C.

7. Process of claim 6, characterized in that the inorganic counterion is preferably selected from sodium, potassium, calcium, magnesium and ammonium.

8. Process of claim 6, characterized in that the low molecular weight heparin is obtained by thermolytic decomposition of an aqueous solution of hypochlorous acid at a temperature from 0 to 5° C. on an aqueous solution of 15-25% concentration of injectable heparin at pH from 4 to 7 and a temperature from 70 to 100° C. at reflux.

9. Process of claim 6, characterized in that the low molecular weight heparin is obtained through the radical decomposition of hydrogen peroxide catalized by ferrous ions on the injectable heparin, being the concentration of ferrous ion from 10 to 100 ppm, the concentration of hydrogen peroxide in a molar ratio of H₂O₂/polysaccharide from 6-15, pH from 4 to 7 and a reaction temperature from 60 to 120° C.

10. Process for eliminating hygroscopicity of the low molecular weight heparin salt with triethanolamine of claim 1, characterized in that it comprises dissolving said salt in water until reaching a concentration of 13 to 17%, adding mannitol or sorbitol in an amount equivalent to 30-50 parts each 60 parts of said salt, dissolving all in aqueous solution and lyophilize.

11. Pharmaceutical compositions for local use in antithrombotic therapy which comprise the low molecular weight heparin salt with triethanolamine of claim 1, characterized in that said salt is present in pharmaceutically active amounts from 100 to 2000 international units Anti X_{A}/g of composition of local use diluted in an adequate excipient.

12. Pharmaceutical compositions of claim 11, characterized in that the excipient is selected from aqueous gels of carbopolymers, creams or emulsions of the oil-in-water type, or ointments of aqueous or hydroalcoholic base.

13. Use of low molecular weight heparin salt with triethanolamine as antithrombotic therapeutic agent of local administration, characterized in that it is used for the treatment, alleviation or prevention of varicose veins, hemorrhoids, traumatic or post-surgical hematomas or phlebitis.

14. Use of claim 13, characterized in that the post-surgical hematoma is derived from a plastic surgery or facial reconstruction.

15. Use of claim 13, characterized in that the phlebitis is caused by the intravenous infusion of chemotherapeutic agents.
